(19) United States
(12) Patent Application Publication
(10) Pub. No.: US 2022/0254481 A1
(43) Pub. Date: Aug. 11, 2022

(54) SYSTEM AND METHOD FOR CUSTOMIZED AUGMENTED REALITY NAVIGATION

(71) Applicant: Surgical Theater, Inc., Los Angeles, CA (US)

(72) Inventors: MORDECHAI AVISAR, HIGHLAND HEIGHTS, OH (US); Alon Yakob Geri, Orange Village, OH (US); Nate REGEV, Beverly Hills, CA (US)

(73) Assignee: SURGICAL THEATER, INC., Los Angeles, CA (US)

(21) Appl. No.: 17/669,032

(22) Filed: Feb. 10, 2022

Related U.S. Application Data
(60) Provisional application No. 63/148,446, filed on Feb. 11, 2021.

(51) Int. Cl. G16H 40/20; G06Q 10/10

(52) U.S. Cl. CPC G16H 40/20; G06Q 10/1095

(57) ABSTRACT

A navigation system that utilizes existing mobile computing and mapping technologies and provides a user with a unique and customized augmented reality navigation experience to support user appointments from an arbitrary starting point into the facility where the user has the appointment and ultimately to the destination appointment room. For medical patients, the system can accommodate medical information and instructions regarding the patient using the system.

### BACKGROUND

This application claims the benefit of U.S. provisional application Ser. No. 63/148,446 filed on Feb. 11, 2021, which is incorporated herein by reference.

When traveling or moving between two geographical points, such as by car, by foot, by bicycle, and so on, one must be aware of the geographical region and surroundings in order to effectively navigate between the two pints. Navigation between two geographic points may be challenging, however, if one is not familiar with the geographic region and surroundings. Mapping technologies and services, such as those developed by Google and Apple for example, enable easy and efficient navigation between a source and a destination.

Recent technological developments have enabled mapping technologies and services to become even more effective and easier to use. For example, with the prevalence of mobile computing devices and smartphones that include GPS tracking capabilities, mapping technologies have been incorporated into the mobile devices in order to provide users with the ability to track location and navigate in real time as they are traveling between the geographical points. Also, advances in tracking capabilities have enabled mobile devices to allow for indoor navigation as well as outdoor navigation. Finally, developments in augmented reality technologies are being incorporated into mobile mapping solutions by superimposing navigation instructions over top of a live view of geographic surroundings using a camera of the mobile device.

### SUMMARY

Provided are a plurality of example embodiments, including, but not limited to, a method of providing, via a mobile device, navigation assistance to a patient having a medical appointment at a medical facility, comprising the steps of: obtaining medical appointment information about the medical appointment of the patient from a database, said medical appointment information including a location of the medical facility and also including a location of a room within the medical facility where the patient is to go for the appointment; maintaining current patient location information of the patient during performance of the method; determine an external navigation path for the patient from the patient's current location to the medical facility; display, using the mobile device, external navigation information to the patient, said external navigation information including images of features found along the external navigation path, wherein said navigation information is updated as the current patient location changes along the external navigation path; when the current patient location information shows that the patient has arrived at the medical facility, display navigation information including images of an entrance to said medical facility; when the current patient location information shows that the patient has neared or entered the medical facility, determine an internal navigation path through the medical facility to the room within the medical facility where the appointment is scheduled; display, using the mobile device, internal navigation information to the patient, said internal navigation information including images of features within the medical facility found along the internal navigation path, wherein said internal navigation information is updated as the current patient location changes along the internal navigation route; and notifying the patient when the patient has arrived at the room within the medical facility where the appointment is scheduled.

Also provided is a method of providing, via a mobile device, navigation assistance to a patient having a medical appointment at a medical facility, comprising the steps of: obtaining medical appointment information about the medical appointment of the patient from a database, said medical appointment information including a location of the medical facility and also including a location of a room within the medical facility where the patient is to go for the appointment; maintaining current patient location information of the patient during performance of the method; determine an external navigation path for the patient from the patient's current location to the medical facility; display, using the mobile device, external navigation information to the patient, said external navigation information including images of features found along the external navigation path, wherein said navigation information is updated as the current patient location changes along the external navigation path; when the current patient location information shows that the patient has arrived at the medical facility, display navigation information including images of an entrance to said medical facility; obtaining patient medical information about the patient from the database; when the current patient location information shows that the patient has neared or entered the medical facility, determine an internal navigation path through the medical facility to the room within the medical facility where the appointment is scheduled, wherein said internal navigation path is determined utilizing said patient medical information to determine the internal navigation path that is optimal for a medical condition of the patient; display, using the mobile device, internal navigation information to the patient, said internal navigation information including images of features within the medical facility found along the internal navigation path, wherein said internal navigation information is updated as the current patient location changes along the internal navigation route; wherein said patient medical instructions for the patient includes instructions for a medical procedure to be performed on the patient prior to arriving at said room; and wherein the internal navigation path includes navigation instructions to a location to perform said procedure; and notifying the patient when the patient has arrived at the room within the medical facility where the appointment is scheduled.

Further provided is method of providing, via a mobile device, navigation assistance to a person traveling to a particular location in a facility for a scheduled appointment, comprising the steps of: obtaining scheduled appointment information about the scheduled appointment from a database, said scheduled appointment information including a location of the facility and also including a location of a room within the facility where the person is to go for the appointment; maintaining current person location information of the person during performance of the method; determine an external navigation path for the person from the person's current location to the facility; display, using the mobile device, external navigation information to the person, said external navigation information including images of features found along the external navigation path, wherein said navigation information is updated as the current person location changes along the external navigation path; when the current person location information shows that the patient has arrived at the facility, display navigation information including images of an entrance to said facility; when the current person location information shows that the person has neared or entered the facility, determine an internal navigation path through the facility to the room within the facility where the appointment is scheduled; display, using the mobile device, internal navigation information to the person, said internal navigation information including images of features within the facility found along the internal navigation path, wherein said internal navigation information is updated as the current person location changes along the internal navigation route; and notifying the person when the person has arrived at the room within the facility where the appointment is scheduled.

Still further provided is a system using a computer system and the remote device for implementing any of the above methods.

Also provided are additional example embodiments, some, but not all of which, are described hereinbelow in more detail.

### BRIEF DESCRIPTION OF THE DRAWINGS

In the accompanying drawings, structures are illustrated that, together with the detailed description provided below, describe exemplary embodiments of the claimed invention. Like elements are identified with the same reference numerals. It should be understood that elements shown as a single component may be replaced with multiple components, and elements shown as multiple components may be replaced with a single component. The drawings are not to scale and the proportion of certain elements may be exaggerated for the purpose of illustration.

FIG. 1 illustrates an example system for customized augmented reality navigation.

FIG. 1A illustrates an example architecture for customized augmented reality navigation.

FIG. 2 illustrates an example screen shot of an AR user interface for providing customized AR navigation.

FIG. 3 illustrates an example screen shot of an AR user interface for providing customized AR navigation.

FIG. 4 illustrates an example screen shot of an AR user interface for providing customized AR navigation.

FIG. 5 illustrates an example screen shot of an AR user interface for providing customized AR navigation.

FIG. 6 illustrates an example screen shot of an AR user interface for providing customized AR navigation.

FIG. 7 illustrates an example screen shot of an AR user interface for providing customized AR navigation.

FIG. 8 illustrates an example screen shot of an AR user interface for providing customized AR navigation.

FIG. 9 illustrates an example screen shot of an AR user interface for providing customized AR navigation.

FIG. 10 illustrates an example method for customized augmented reality navigation.

FIG. 11 illustrates an example computer implementing the example mobile computing device and the AR computer computer of FIG. 1.

### DETAILED DESCRIPTION

The Wayfinder system described herein leverages existing mobile computing and mapping technologies and provides a user with a unique and customized augmented reality navigation experience, thus creating a more relevant, engaging and satisfying experience for the user. In particular, the Wayfinder system integrates with resources that provide user specific information with navigation resources and destination specific resources, and leverages those resources in order to customize a navigation experience according to user-specific needs and with user specific content.

It should be appreciated that although specific references to healthcare applications and patient navigation and engagement will be made through out the examples described herein, the Wayfinder system may also be used to provide any types of users with unique and customized augmented reality navigation experiences in a variety of applications and settings. For example, the Wayfinder may be used to provide a shopper with a customized experience inside a shopping mall or a student with a customized experience inside a school.

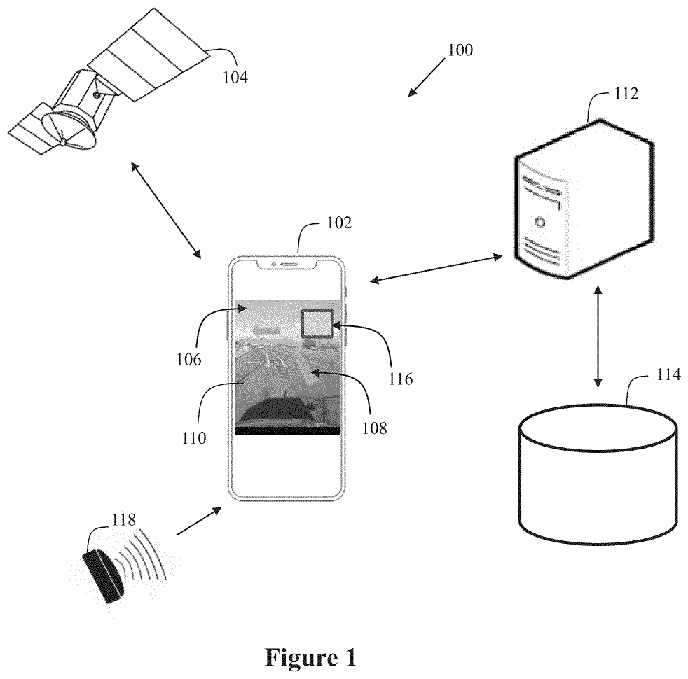

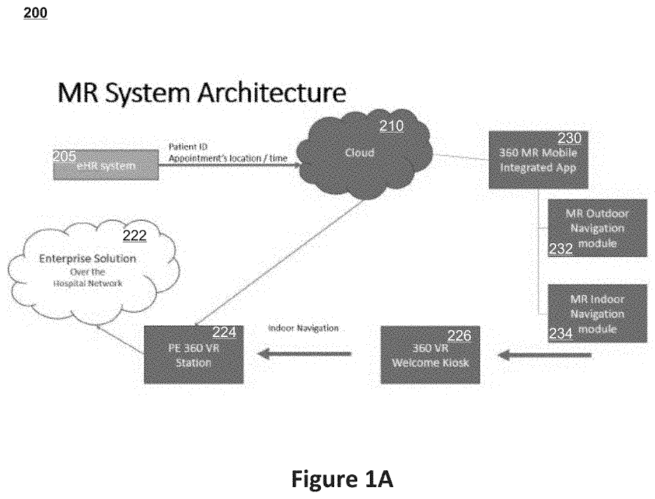

FIG. 1 illustrates an example customized augmented reality navigation system 100. The system 100 includes a navigation computing device 102, which may be a smart phone of the patient, or a tablet or other device that is provided to the patient, that is configured to communicate with a GPS satellite 104 in order to obtain GPS data and to use the GPS data to provide navigation instructions via user interface 106. The device might also, or alternatively, communicat with cell phone towers or other resources to aid in detecting the location of the device. In particular, the navigation computing device 102 determines a path between a starting geographic location, such as the home of a patient, and a destination, such as an appointment room, using the GPS or other location data and by connecting to a mapping solution such as Google Maps or proprietary navigation maps or other mapping resources, and provides navigation instructions 108 visually by overlaying the instructions in augmented reality over top of a real time view and images of the surrounding geography, which may include landmarks, road signs, landscapes, etc., along with the names of roads, paths, landmarks, or other objects found along the navigation path. In other words, as a user of the navigation computing device 102 travels along a path 110, navigation instructions 108, such as an arrow indicating a direction to turn, is overlaid on top of the path 110. It should be appreciated that although the example navigation computing device 102 is illustrated as a smartphone, it can take the form of any suitable computing device with AR capabilities. In one example, the navigation computing device 102 includes AR glasses.

The system 100 further includes an augmented reality navigation computer (“AR computer”) 112, with which the navigation computing device 102 is configured to communicate with and to receive customized content from based on a user's identify, preferences, and other relevant factors and data. More specifically, the AR computer 112 is configured to retrieve data from a resource 114, such as a data store, based on the identity of the user and creates custom content for the user. Resources that may be accessed include internet sits, public and/or proprietary databases, etc. The AR computer 112 is further configured to feed the custom content to the navigation computing device 102 so that the navigation computing device 102 may generate augmented custom content 116 and overlay it on top of the view of the path 110.

The system 100 further includes one or more beacons 118 that may be positioned inside a building or structure such as a hospital, shopping mall, school and so on in order to facilitate indoor navigation in addition to outdoor navigation. In particular, the beacons 118 communicate wireless signals to the navigation computing device 102 which in turn leverage the beacon's 118 signals to determine position inside the building and to facilitate navigation inside the building. Furthermore, the system 100 may include one or more computers and/or databases found at the destination location that store information useful for navigating the destination location, including images of internal structures like doors, elevators, signage, pathways, rooms, etc. Thus, the mobile computing device 102 includes both an outdoor navigation module and an indoor navigation module (not shown) that are configured to interact and communicate with one another and to easily transition navigation responsibilities between the two modules depending on a current location of a user.

By having both indoor and outdoor tracking capabilities and being able to automatically transition between the two, the system 100 enables seamless navigation and transition when a user moves from outdoors (following an external navigation path to arrive at the builiding) to inside the building (and traversing an internal navigation path through the building to the ultimate destination(s)). For example, the navigation computing device 102 can assist the user in navigating to the destination building from an origin such as a home, office, or arbitrary location, to arrive at the destination and, if driving a vehicle, to parking locations, and then continue to assist the user to navigate into the building by a preferred or appropriate entrance and to a specific location or series of locations inside the building seamlessly. Although indoor navigation capabilities are described as being implemented using beacons 118, the system 100 may also leverage other known and suitable technologies or indoor positioning systems (“IPS”), such as visual positioning, visual markers, GPS, WiFi, etc. to determine locations and to measure distances between locations and nodes in order to facilitate indoor navigation. The nodes either actively locates the mobile computing device 102 or provides ambient location or environmental context for the mobile computing device 102 to get sensed.

The resource 114 may be any suitable public or private resource 114, database, content repository, etc. that may include information specific about the user or even general information that may be retrieved and tailored specifically for the user. For example, for a patient visiting a medical facility, information about the users medical condition, medical appointments, doctor prescriptions and orders, etc. may all be stored and made available. As another example, the class schedule and classroom locations, and mobility capabilities of a student can be stored and made available. The information or content retrieved may then be used by the navigation computing device 102 to create a customized navigation experience for the user. For example, the navigation computing device 102 may navigate the user directly to a position inside a building depending the user's specific need for traveling to the building. The navigation computing device 102 may also make customized recommendations along the way for additional stops, either outside or inside the building, depending on the user's specific need for traveling to the building, the user's history, and other personal factors obtained from the resource 114, and adjust the navigation instructions accordingly.

In one example, the resource 114 may include shopping data about a user. In such an example, as the navigation computing device 102 is navigating the shopper to a store, for example, the navigation computing device 102 may make customized recommendations along the path for the shopper to consider stopping at other stores along the path which may offer complimentary products or special sales customized for the specific shopper.

In another example, the resource 114 may include school records. In such an example, the navigation computing device 102 may assist the student with navigating both to a school as well as to specific classrooms inside the school based on the specific student's course schedule. The navigation computing device 102 may also provide customized recommendations for the student to stop along the way to the school to buy school supplies, for example, based on the student's specific schedule and supply needs. The navigation computing device 102 may also make recommendations based on the student's schedule while navigating inside the school to a classroom, such as to stop and use a restroom or to buy a snack in between classes if a schedule is such that a snack break or a bathroom brake may be difficult to fit in later.

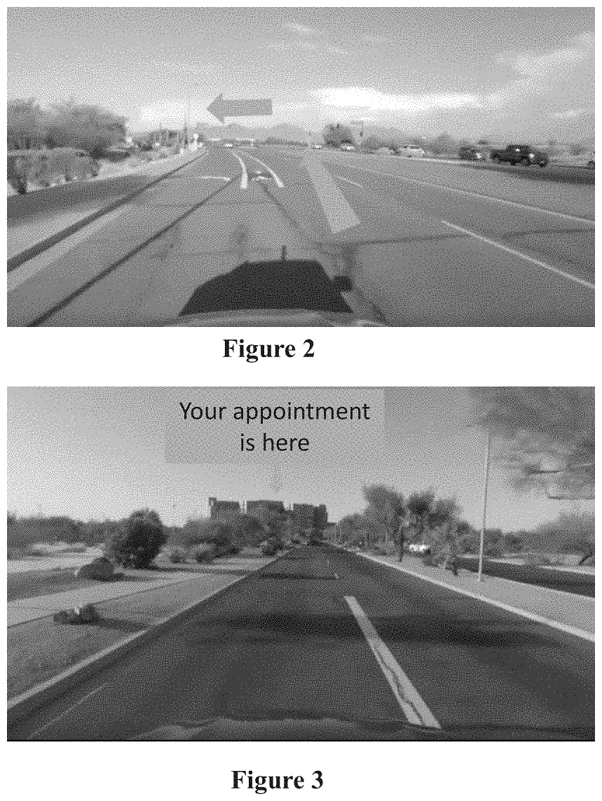

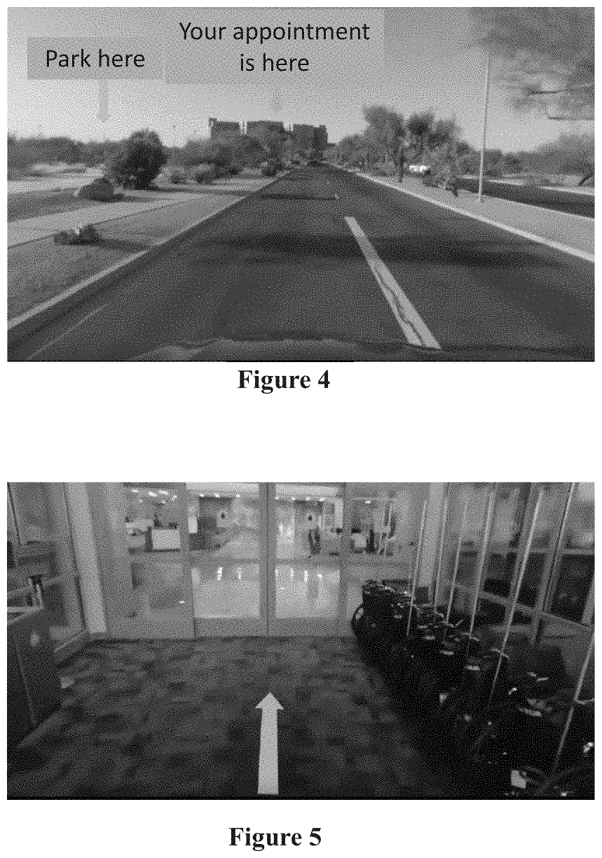

In another example, the resource 114 may be an electronic health records (“EHR”) database and include medical records and other medical information (in compliance with HIPAA) about a patient such as information about a patient's next medical appointment, prescriptions, doctors' orders and treatments, etc. In such an example, the navigation computing device 102 may provide an external navigation path to help navigate the patient from his home, office, or other starting location to a hospital or other medical facility at which his next appointment is scheduled. The navigation computing device 102 may also seamlessly switch to indoor navigation and help direct the patient to the specific doctor's office within the hospital as the patient enters the medical facility. In addition, the navigation computing device 102 may make customized recommendations for the patient along the navigated path based on the patient's medical history and doctors orders and/or treatments. For example, if the EHR record indicates that the patient has not yet received a flu shot, the navigation computing device 102 may suggest, as the patient is walking through the hospital to reach his final destination of his doctor's office for his appointment, that the patient also stop and get a flu shot along the same path (or maybe make a slight detour from the path). As another example, the patient may need to get blood work done, or medical imaging such as x-rays or MRIs or CT scans, prior to, or subsequent to, meeting the doctor. FIGS. 2-9 illustrate example screen shots of the example user interface 106 for navigating a patient to a hospital and within the hospital as provided by the navigation computing device 102 and powered by the AR computer 112.

FIG. 1A shows an example system architecture 200 for navigation system 205, utilizing cloud-based services 210 to support a mobile application 230 including an outdoor navigation module 232 for supporting external navigation, and an indoor navigation module 234 for supporting indoor navigation, on a mobile device. The mobile device can interact with a local VR stations 234 and welcome kiosks 226 operating in connection with an enterprise cloud 222 for the facility. The system can be adapted for use with any types of mobile devices, such as using an Android or iOS platform, for example.

FIG. 2 illustrates an example screen shot of an AR user interface navigating a user to a destination, in this example to a hospital for a scheduled medical appointment. The arrows in yellow provide the user with guidance and directions in order to reach the destination. Although yellow arrows are used in the example interface, any suitable instructions or guidance may be overlaid in AR on top of the road to help guide the user to the hospital. As illustrated in FIG. 2, when the patient approaches the hospital at which the medical appointment is scheduled, an indication is provided to notify the patient and to confirm the patient is in the correct location. In one example, a recommendation for where to park may also be provided to the patient as the patient approaches the hospital. The parking recommendation may be made based on data retrieved from a resource 114. For example, the parking recommendation may be made based on the location of the doctor's appointment within the hospital or the patient's medical condidition (e.g., handicapped status) so as to minimize the distance the patient will be required to walk from the car. In another example, the parking recommendation may be made based on availability of parking spaces as tracked by the resource 114.

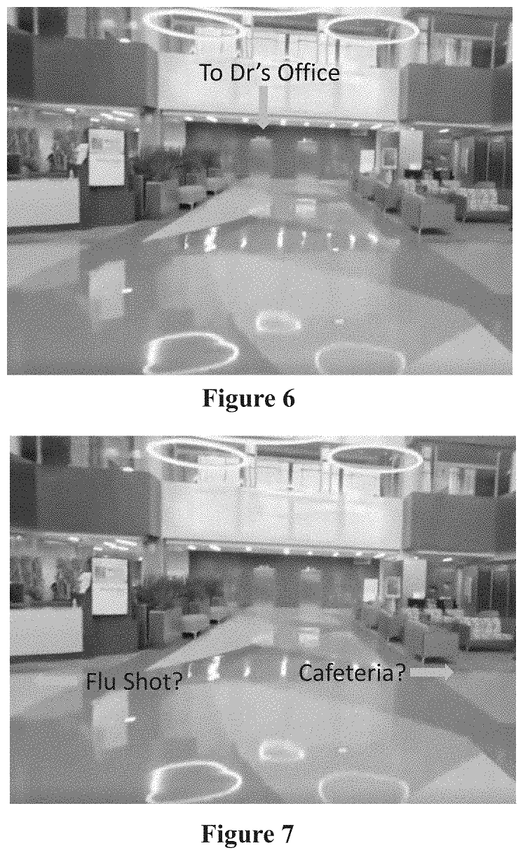

Once the patient parks the car, the navigation assistance continues by providing an internal navigation path, as illustrated in FIG. 5, by directing the patient to the entrance of the hospital. An indoor Positionint System (IPS) may be provided or utilized for this purpose. In one example, the patient may be directed to an entrance selected from multiple entrances based on proximity to the location of the patient's appointment within the hospital. Once inside the hospital, as illustrated in FIG. 6, the patient is directed to a doctor's office specific to the patient's scheduled appointment. For example, a patient may have an appointment scheduled with a doctor on the second floor in office number 200. Thus, the patient may be directed to go to the elevator. In one example (not shown), once inside the elevator, an arrow may point to button #2 on the button panel instructing the patient to select the “2^{nd}” floor. In one example, as illustrated in FIG. 7, a recommendation may be made for the patient to take a detour and go to the left in order to first get a flu shot if the patient's EHR indicates that the patient has not yet had a flu shot. In another example, a recommendation may be made for the patient to turn right and get a snack in the cafeteria before proceeding to the scheduled appointment. In one example, the recommendation to go to the cafeteria may be made based on information obtained regarding the current wait time at the doctor's office. In other words, if the doctor which the patient is scheduled to see is running behind schedule and the patient will need to wait an extra 30 minutes, it may be suggested to the patient to visit the cafeteria first in order to improve his satisfaction and experience at the hospital. Computer resources at the facility can be used to provide images and internal landmarks to ease the patient's pathway through the facility.

At all times, the location of the patient can be monitored and provided to the system to aid in the navigation activity. For example, GPS information and/or cell tower location information can be provided by a mobile device carried by the patient, such as a smartphone, for example, when the patient is navigation the external navigation path. When the patient nears or enters the destination facility, internal location beacons, GPS, mobile device monitoring devices, or other means of information location can be used to monitor the current location of the patient at all times inside the facility. The indoor Positionint System (IPS) may be provided or utilized for this purpose, which can provide technologies like distance measures to nearby anchor nodes to determine locations, for example. Such nodes either actively locate mobile devices and tags, or provide ambient location or environmental context for devices to get sensed

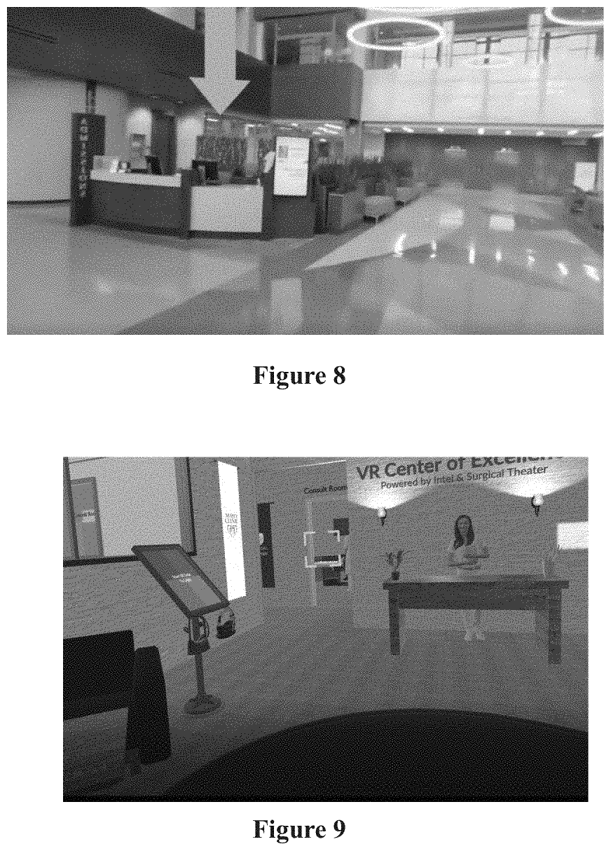

In one example, as illustrated in FIG. 8, the patient may be directed to an information kiosk upon entering the hospital. In one example, a welcome video or other instructional video may be displayed in AR to the patient upon the patient reaching the welcome kiosk, or other predetermined location. In one example, a virtual assistant may appear in AR to the patient upon the patient reaching the kiosk or other location, with which the patient may be able to interact and ask questions. In one example, in order to connect with the hospital's EHR and to receive a customized patient specific AR navigation experience, the patient may be required to first log in or sign in at the kiosk using a QR code, a fingerprint scan, an eye scan, or using any suitable means for authenticating the identity of the patient.

FIG. 9 illustrates an example screen shot of the user interface upon the patient entering the doctor's office. The patient may be greeted by a virtual assistant. In one example, the virtual assistant may ask the patient to have a seat and wait in a sitting area. In one example, the patient may be directed, using navigation instructions overlaid within the user interface display, to a specific exam room within the doctor's office.

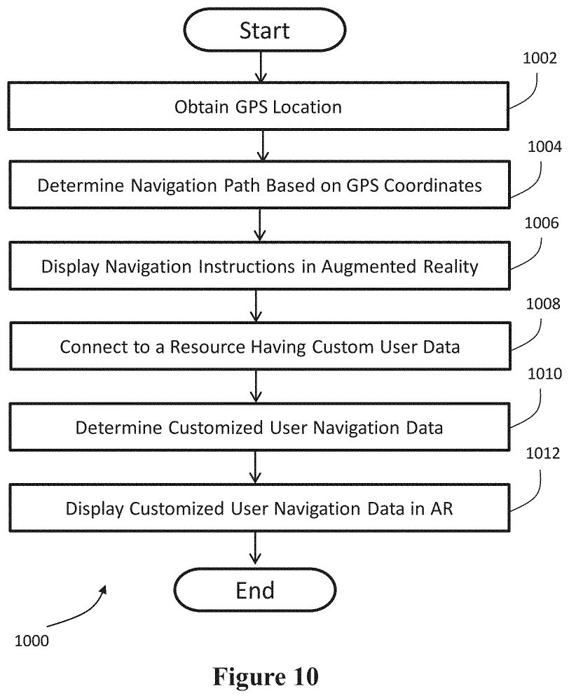

FIG. 10 illustrates an example method for customized augmented reality navigation. At 1002, a mobile computing device 102 obtains GPS coordinates of a current location from a GPS satellite 104. At 1004, the mobile computing device 102 connects to a mapping solution, such as Google Maps, and determines a navigation path from a starting location to a destination. At 1006, the mobile computing device 102 communicates the navigation instructions to a user via an augmented reality user interface by overlaying navigation instructions over top of a real time view of the road or path in front of the user.

At 1008, the mobile computing device 102, via an AR computer 112, connects to a resource 114 having custom user data. At 1010, the mobile computing device 102, via the AR computer 112, determines customized user navigation data based on the identification of the user and the data retrieved from the resource 114. At 1014, the mobile computing device 102 communicates the customized navigation data to the user via the augmented reality user interface by overlaying the customized navigation data alongside the navigation instructions over top of the real time view of the road or path in front of the user.

Provisional application Ser. No. 63/148,446 filed on Feb. 11, 2021, provides an Appendix, incorporated herein by reference, that includes diagrams and descriptions providing an example Mixed Reality (MR) System Architecture; Connectivity to EHR; a Cloud architecture; an Integration App; MR Outdoor Navigation; Indoor navigation; and an ST 360 VR Welcome Kiosk;

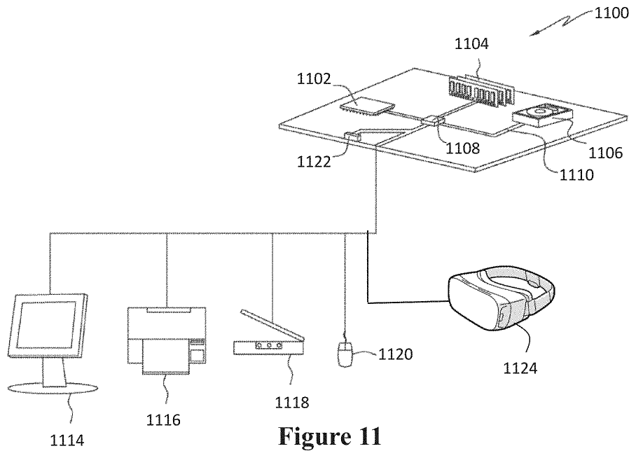

FIG. 11 is a schematic diagram of an example computer for implementing the mobile computing device 102 and the AR computer 112 of FIG. 1. The example computer 1100 is intended to represent various forms of digital computers, including laptops, desktops, handheld computers, tablet computers, smartphones, servers, AR glasses, and other similar types of computing devices. Computer 1100 includes a processor 1102, memory 1104, a storage device 1106, and a communication port 1108, operably connected by an interface 1110 via a bus 1112.

Processor 1102 processes instructions, via memory 1104, for execution within computer 1100. In an example embodiment, multiple processors along with multiple memories may be used.

Memory 1104 may be volatile memory or non-volatile memory. Memory 1104 may be a computer-readable medium, such as a magnetic disk or optical disk. Storage device 1106 may be a computer-readable medium, such as floppy disk devices, a hard disk device, optical disk device, a tape device, a flash memory, phase change memory, or other similar solid state memory device, or an array of devices, including devices in a storage area network of other configurations. A computer program product can be tangibly embodied in a computer readable medium such as memory 1104 or storage device 1106.

Computer 100 can be coupled to one or more input and output devices such as a display 1114, a printer 1116, a scanner 1118, a mouse 1120, and a HMD 1124.

As will be appreciated by one of skill in the art, the example embodiments may be actualized as, or may generally utilize, a method, system, computer program product, or a combination of the foregoing. Accordingly, any of the embodiments may take the form of specialized software comprising executable instructions stored in a storage device for execution on computer hardware, where the software can be stored on a computer-usable storage medium having computer-usable program code embodied in the medium.

Databases may be implemented using commercially available computer applications, such as open source solutions such as MySQL, or closed solutions like Microsoft SQL that may operate on the disclosed servers or on additional computer servers. Databases may utilize relational or object oriented paradigms for storing data, models, and model parameters that are used for the example embodiments disclosed above. Such databases may be customized using known database programming techniques for specialized applicability as disclosed herein.

Any suitable computer usable (computer readable) medium may be utilized for storing the software comprising the executable instructions. The computer usable or computer readable medium may be, for example but not limited to, an electronic, magnetic, optical, electromagnetic, infrared, or semiconductor system, apparatus, device, or propagation medium. More specific examples (a non-exhaustive list) of the computer readable medium would include the following: an electrical connection having one or more wires; a tangible medium such as a portable computer diskette, a hard disk, a random access memory (RAM), a read -only memory (ROM), an erasable programmable read-only memory (EPROM or Flash memory), a compact disc read-only memory (CDROM), or other tangible optical or magnetic storage device; or transmission media such as those supporting the Internet or an intranet.

In the context of this document, a computer usable or computer readable medium may be any medium that can contain, store, communicate, propagate, or transport the program instructions for use by, or in connection with, the instruction execution system, platform, apparatus, or device, which can include any suitable computer (or computer system) including one or more programmable or dedicated processor/controller(s). The computer usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, local communication busses, radio frequency (RF) or other means.

Computer program code having executable instructions for carrying out operations of the example embodiments may be written by conventional means using any computer language, including but not limited to, an interpreted or event driven language such as BASIC, Lisp, VBA, or VBScript, or a GUI embodiment such as visual basic, a compiled programming language such as FORTRAN, COBOL, or Pascal, an object oriented, scripted or unscripted programming language such as Java, JavaScript, Perl, Smalltalk, C++, C#, Object Pascal, or the like, artificial intelligence languages such as Prolog, a real-time embedded language such as Ada, or even more direct or simplified programming using ladder logic, an Assembler language, or directly programming using an appropriate machine language.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative apparatus and method, and illustrative examples shown and described. Accordingly, departures may be made from such details without departing from the spirit or scope of the applicant's general inventive concept.

What is claimed is:

1. A method of providing, via a mobile device, navigation assistance to a patient having a medical appointment at a medical facility, comprising the steps of:
obtaining medical appointment information about the medical appointment of the patient from a database, said medical appointment information including a location of the medical facility and also including a location of a room within the medical facility where the patient is to go for the appointment;
maintaining current patient location information of the patient during performance of the method;
determine an external navigation path for the patient from the patient's current location to the medical facility;
display, using the mobile device, external navigation information to the patient, said external navigation information including images of features found along the external navigation path, wherein said navigation information is updated as the current patient location changes along the external navigation path;
when the current patient location information shows that the patient has arrived at the medical facility, display navigation information including images of an entrance to said medical facility;
when the current patient location information shows that the patient has neared or entered the medical facility, determine an internal navigation path through the medical facility to the room within the medical facility where the appointment is scheduled;
display, using the mobile device, internal navigation information to the patient, said internal navigation information including images of features within the medical facility found along the internal navigation path, wherein said internal navigation information is updated as the current patient location changes along the internal navigation route; and
notifying the patient when the patient has arrived at the room within the medical facility where the appointment is scheduled.

2. The method of claim 1, further comprising the steps of:
obtaining patient medical information about the patient from the database; and
utilizing said patient medical information to determine the internal navigation path that is optimal for a medical condition of the patient.

3. The method of claim 2, wherein said patient medical information about the patient includes information about a handicap of the patient; and wherein the internal navigation path that is optimal for the medical condition of the patient includes adjusting the navigation path to accommodate the handicap of the patient.

4. The method of claim 1, further comprising the steps of:
obtaining patient medical information about the patient from the database; and
utilizing said patient medical information to determine the external navigation path that is optimal for a medical condition of the patient.

5. The method of claim 4, wherein adjusting the navigation path to accommodate the handicap of the patient includes utilizing handicap parking and/or handicap entrances to the medical facility.

6. The method of claim 1, further comprising the steps of:
obtaining medical instructions for the patient from the database; and
utilizing said doctor instructions to determine the internal navigation path that accommodates the medical instructions for the patient.

7. The method of claim 6, wherein said patient medical instructions for the patient includes instructions for a medical procedure to be performed on the patient prior to arriving at said room; and wherein the internal navigation path includes navigation instructions to a location to perform said procedure.

8. The method of claim 7, wherein said medical procedure includes taking blood from the patient.

9. The method of claim 7, wherein said medical procedure includes taking x-rays of the patient.

10. The method of claim 7, wherein said procedure includes removing a wound dressing of the patient.

11. The method of claim 1, wherein said images of features within the medical facility are stored in a database of the medical facility and are transmitted to the mobile device by a transmitting device of the medical facility.

12. The method of claim 1, wherein said images of the entrance to said medical facility are selected based on a location the patient has parked a vehicle in a parking location of the medical facility.

13. The method of claim 1, wherein the step of maintaining current patient location information of the patient during performance of the method includes using beacons broadcast by the medical facility when the patient is inside said medical facility traversing said internal navigation path.

14. The method of claim 1, wherein said internal navigation information displayed to the patient includes displaying a direction indicator with said images of features within the medical facility to show the patient a direction to travel along said internal navigation path.

15. The method of claim 14, wherein said direction to travel includes the direction indicator showing a door to enter.

16. The method of claim 14, wherein said direction to travel includes the direction indicator showing an elevator to enter.

17. The method of claim 16, wherein the mobile device indicates to the patient a button to push on the elevator.

18. The method of claim 1, wherein said external navigation information displayed to the patient includes information showing the patient where to park a vehicle along the internal navigation path.

19. The method of claim 18, wherein said information showing the patient where to park a vehicle along the internal navigation path provides an optimal location for the patient to park.

20. The method of claim 1, wherein said mobile device transmits information to the medical facility notifying that the patient has arrived at the medical facility.

21. The method of claim 1, wherein said internal navigation path includes providing a waiting area when there is a waiting period to see a medical personnel for the medical appointment.

22. The method of claim 1, wherein said internal navigation path includes a stop at an information station in the medical facility.

23. The method of claim 1, further comprising a step of displaying an informational video to the patient on the medical device when the patient reaches a particular location along the internal navigation path.

24. The method of claim 1, further comprising a step of providing instructions to the patient using the medical device when the patient reaches a particular location along the internal navigation path.

25. A method of providing, via a mobile device, navigation assistance to a patient having a medical appointment at a medical facility, comprising the steps of:
obtaining medical appointment information about the medical appointment of the patient from a database, said medical appointment information including a location of the medical facility and also including a location of a room within the medical facility where the patient is to go for the appointment;
maintaining current patient location information of the patient during performance of the method;
determine an external navigation path for the patient from the patient's current location to the medical facility;
display, using the mobile device, external navigation information to the patient, said external navigation information including images of features found along the external navigation path, wherein said navigation information is updated as the current patient location changes along the external navigation path;
when the current patient location information shows that the patient has arrived at the medical facility, display navigation information including images of an entrance to said medical facility;
obtaining patient medical information about the patient from the database;
when the current patient location information shows that the patient has neared or entered the medical facility, determine an internal navigation path through the medical facility to the room within the medical facility where the appointment is scheduled, wherein said internal navigation path is determined utilizing said patient medical information to determine the internal navigation path that is optimal for a medical condition of the patient;
display, using the mobile device, internal navigation information to the patient, said internal navigation information including images of features within the medical facility found along the internal navigation path, wherein said internal navigation information is updated as the current patient location changes along the internal navigation route;
wherein said patient medical instructions for the patient includes instructions for a medical procedure to be performed on the patient prior to arriving at said room; and
wherein the internal navigation path includes navigation instructions to a location to perform said procedure; and
notifying the patient when the patient has arrived at the room within the medical facility where the appointment is scheduled.

26. A system including a computer system and a mobile device, said system configured to provide navigation assistance to a patient having a medical appointment at a medical facility, comprising:
a computer system configured to store and retrieve medical appointment information about the medical appointment of the patient from a database, said medical appointment information including a location of the medical facility and also including a location of a room within the medical facility where the patient is to go for the appointment;
obtaining patient medical information about the patient from the database; and
utilizing said patient medical information to determine the internal navigation path that is optimal for a medical condition of the patient
the system being configured to access current patient location information of the patient while the patient is in transit to, and in, the medical facility;
the system being configured to determine an external navigation path for the patient from the patient's current location to the medical facility;
the mobile device having a display, said mobile device configured for displaying external navigation information to the patient, said external navigation information including images of features found along the external navigation path, wherein said navigation information is updated as the current patient location changes along the external navigation path;
the mobile device being configured to display navigation information including images of an entrance to said medical facility when the current patient location information shows that the patient has arrived at the medical facility;
the system being configured to determine an internal navigation path through the medical facility to the room within the medical facility where the appointment is scheduled when the current patient location information shows that the patient has neared or entered the medical facility;
the mobile device being configured to display internal navigation information to the patient, said internal navigation information including images of features within the medical facility found along the internal navigation path, wherein said internal navigation information is updated as the current patient location changes along the internal navigation route; and
the system being configured to use the mobile device to notify the patient when the patient has arrived at the room within the medical facility where the appointment is scheduled.
